(19) United States
(12) Patent Application Publication
(10) Pub. No.: US 2025/0306151 A1
(43) Pub. Date: Oct. 2, 2025

(54) TECHNIQUE FOR DETERMINING PHYSIOLOGICAL SIGNALS USING MRI SCANS

(71) Applicant: Siemens Healthineers AG, Forchheim (DE)

(72) Inventor: Mario Zeller, Erlangen (DE)

(21) Appl. No.: 19/090,955

(22) Filed: Mar. 26, 2025

(30) Foreign Application Priority Data
Mar. 28, 2024 (DE) 10 2024 202 961.0

(51) Int. Cl. G01R 33/56; A61B 5/00; A61B 5/055

(52) U.S. Cl. CPC G01R 33/5608; A61B 5/055; A61B 5/7267; A61B 5/7289

(57) ABSTRACT

A computer-implemented method for determining a physiological signal of a patient using a magnetic resonance imaging, MRI, scan. The method includes a step of receiving raw data of an MRI scan of a patient. The method further comprises a step of determining, by a neural network, a physiological signal of the patient from the received raw data. The neural network includes a transformer architecture.

### CROSS REFERENCE TO RELATED APPLICATIONS

This application claims the benefit of DE 10 2024 202 961.0 filed on Mar. 28, 2024, which is hereby incorporated by reference in its entirety.

### FIELD

Embodiments relate to a technique for determining a physiological signal of a patient using a magnetic resonance imaging (MRI) scan.

### BACKGROUND

Physiological signals, such as a respiratory curve or electrocardiogram (ECG) curve are necessary in MRI imaging for numerous acquisition methods in order to reduce a negative influence on the image quality. For this, the imaging is conventionally triggered using characteristic points in the curves, for example only at instants of shallow breathing or in specific cardiac phases. Additional sensors are customarily necessary in order to acquire the physiological signals. These either have to be laboriously placed on the patient first (for example, ECG electrodes) or are only available in specific constellations. For example, pilot tone transmitters or respiratory sensors are installed only in specific coils.

Further, separate measurement of the physiological signals necessitates computer resource-intensive and time-intensive post-processing of an MRI scan with associated limited accuracy, for example on the basis of an algorithm which customarily quickly forgets in view of fluctuations of the physiological signal which cause short-term disruptions in the measuring procedure to result in serious errors in the post-processing.

### BRIEF SUMMARY AND DESCRIPTION

The scope of the present invention is defined solely by the appended claims and is not affected to any degree by the statements within this summary. The present embodiments may obviate one or more of the drawbacks or limitations in the related art.

Embodiments provide reliable or accurate determination of a physiological signal during acquisition of an MRI scan without additional sensors and/or provide a technique in order to modify raw data of an MRI scan using a physiological signal. Embodiments further facilitate a patient handling and/or to save resources (for example, with regard to material and/or manufacturing) of suitable apparatuses, for example without reducing the accuracy of the MRI data that is obtained.

Embodiments provide a method for determining a physiological signal of a patient using an MRI scan, by a neural network (NN), by a system comprising the NN, by a computer program (and/or a computer program product) and by a computer-readable storage medium.

Embodiments are described below in relation to the method for determining a physiological signal of a patient using a MRI scan and in relation to the neural network. Features, advantages or alternative embodiments herein may be associated with the other subject matters (for example, the computer program or a computer program product), and vice versa. In other words, the embodiments for the neural network may be improved by features which are described or claimed in conjunction with the method, and vice versa. In this case the functional features of the method are configured by structural units of the neural network, and vice versa.

According to one method aspect, a computer-implemented method for determining a physiological signal of a patient using a magnetic resonance imaging (MRI) scan is provided. The method includes the step of receiving raw data of an MRI scan of a patient. The method further includes the step of determining, by a neural network, a physiological signal of the patient from the received raw data. The neural network includes a transformer architecture.

By this technique it is possible to sometimes determine different physiological signals (for example, respiratory curves, electrocardiogram curves and/or movement curves) of a patient during an MRI scan (also: MRI measurement and/or MRI), for example without additional sensors being required. This makes simplified patient handling possible and may prevent errors owing to incorrect sensor measurements.

Alternatively, or in addition, disruptive effects of the MRI scan from different sources may be eliminated and/or patterns in the course over time of the raw data may be identified (and/or utilized). For example, a magnetic field drift (in technical terminology: B0 drift) and/or a phase drift may be compensated. Due to heating of the magnets of the MRI scanner owing to scan activities, for example the basic magnetic field (also: basic B0 field) may shift over the course of a measurement. Alternatively, or in addition, for example the breathing of the patient may influence the phase. For example, this may be an effect on the minute timescale.

Alternatively, or in addition, measurement results of the MRI scan may be adjusted by the technique, for example for improved comparison with standard measurement results and/or measurement results in a predetermined physiological state. Furthermore, alternatively, or in addition, an analysis of the measurement data may be triggered using characteristic points in a physiological signal curve. Triggering may, for example, cause the measurement data to be analyzed only at instants of shallow breathing, of particular cardiac phases (for example, systole and/or diastole) and/or, while the patient lies as still as possible.

The transformer architecture is suitable, for example, for improving a result of determining of the physiological signal (and/or modifying of the MRI raw data) by using historical data about a longer period (for example, numerous breathing cycles and/or cardiac cycles), by weighting data such that short-term disturbances (for example, due to movements of the patient) are negligible and/or by enriching a conventional positional encoding with additional items of information (for example, with regard to slice position, temporal sequence and/or context).

The raw data of the MRI scan (MRI raw data for short) may be complex (and/or have a real part and an imaginary part). Alternatively, or in addition, the MRI raw data may be encoded, for example independently in the imaginary part and in the real part. For example, the instant in the MRI scan may be encoded in the real part (for example, as a, for example conventional, positional encoding. A slice position within a slice stack of the MRI scan may be encoded in the imaginary part. For example, the slice stack may include ten (10) slices.

The MRI raw data may include k-space data, for example, temporally ordered k-space lines. For example, the MRI raw data may include 128 k-space lines per slice and 256 complex-valued samples each. The slice position may be added, for example as a constant offset, to a positional encoding (for example, relative to the first slice of the slice stack) and/or be appended as an additional vector element. For example, 257 complex-valued samples may thus result, of which 256 are associated with positional encodings (for example as measuring instants of the MRI scan) and one with the slice position. Alternatively, or in addition, the original 256 complex-valued samples may be expanded from two channels each to four channels each by the (for example conventional) positional encodings in, for example, the first two channels and the slice position in, for example, the two last channels.

Alternatively, or in addition to the slice position within the slice stack, an acquired contrast (for example in the case of multi-contrast scans, for example by Dixon imaging and/or mapping sequences), an echo train, physiological signals from (for example separate) sensor data and/or a detected movement (for example, from data of a motion sensor) may be encoded.

Dixon imaging exploits the fact that water molecules and fat molecules precess at different frequencies and alternate over time between in-phase and out-of-phase. It is possible to separate water images and fat images by addition and subtraction of the in-phase and out-of-phase measurements.

The mapping sequences take advantage of the fact that the longitudinal T1 relaxation time and the transversal T2 relaxation time (for example depending on weighting) determine an image contrast of the MRI scan.

In the case of multi-contrast scans, for example T1-weighted MRI sequences (T1w), T2-weighted MRI sequences (T2w), T2*-weighted MRI sequences (T2*w), diffusion-weighted MRI sequences (DWI) and/or dynamic contrast agent-enhanced MRI sequences (DCE) may be combined with one another.

Alternatively, or in addition, the MRI raw data may include Fourier transforms of the k-space lines in the readout direction.

Within the context of this application, the term “the physiological signal” is used in the singular. Reference is expressly made to the fact that the term should be taken to mean “at least one physiological signal”. The physiological signal is a signal of the patient, from which the MRI scan originates. The physiological signal relates, for example to an acquisition timeframe (timeframe for short), in which the MRI scan has been captured. The physiological signal may be a signal which changes over an acquisition timeframe, for example is approximately cyclical (for example with a plurality of cycles during the acquisition timeframe). The physiological signal may include, for example, a profile of respiration (also: respiration curve). Alternatively, or in addition, the physiological signal may include a profile of a cardiac function (also: electrocardiogram curve) and/or a movement.

Conventionally, the electrocardiogram curve may be recorded, for example, by an electrocardiogram (ECG). Alternatively, or in addition, the respiration curve may conventionally be acquired, for example, by a respiratory sensor (for example, installed in a spine coil of an MRI scanner). By the described technique it is possible to dispense with additional sensors of this kind—at least in the inference phase of the technique—for recording the respiration curve and/or the electrocardiogram curve. Alternatively, or in addition, it is possible to dispense with additional sensors for measuring a movement of the patient.

The embodiments may include deep learning (DL).

The (for example sequence-to-sequence and/or encoder-decoder) transformer architecture may include at least one encoder and (for example at least) one decoder. The at least one encoder and the (for example at least one) decoder may be separable. For example, the transformer architecture may include an encoder (also: raw data encoder) which uses the received MRI raw data as input data. In one embodiment, the transformer architecture may include a second encoder (also: sensor signal encoder) which includes physiological signals that are received from a sensor (for example during the MRI acquisition timeframe). The second encoder may be used, for example, in a training phase of the transformer architecture. For example, context vectors may be used as output signals of the two encoders (for example for identical positional embedding and slice position) to train the (for example, first) or the encoder for processing the MRI raw data.

A loss function for generating the context vectors may be selected such that the two encoders generate similar embeddings for matching (also: positive and/or corresponding) pairs, for example by maximizing the scalar product of the two vectors for pairs (for example, MRI raw data and during acquisition of the same measured physiological signals) and by minimizing for other combinations (for example, including physiological signals which were not measured during the same acquisition timeframe of the MRI raw data). The embeddings may be generated as a dense representation of the input data.

A context vector may be, for example, between 128 bytes and 4096 bytes long.

A context vector is associated with each instant of the input data of an encoder of the transformer architecture. For the sake of brevity, “context vector” will denote the temporal sequence of all context vectors which pertain to an MRI scan or to a measured physiological signal over an acquisition timeframe (unless something to the contrary emerges from the details of the description).

Correlations over long periods of time may be identified by the transformer architecture. For example, a historical trend may be identified in a respiration curve and/or a cardiac curve (for example over a plurality of breathing cycles or cardiac cycles).

Alternatively, or in addition, a significance of parts of the MRI raw data (and/or of parts of a sequence) may be weighted differently and adapted by self-attention mechanisms in the encoder(s) and in the decoder of the transformer architecture. For example, parts of the MRI raw data, that are briefly disrupted owing to a movement of the patient, may be weighted as unimportant and further parts of the MRI raw data without (or with slight) disruption may be weighted as important.

Furthermore, alternatively or in addition, the conventional positional embeddings (and/or positional encodings), that in each case are fed into one input layer of encoder and decoder, may advantageously be easily expanded in order to encode further relevant data and correlations (for example, a slice position). Determination of the physiological signal from the MRI raw data and/or a corresponding modification (and/or correction) of the MRI raw data may consequently be improved.

The context vector output by an encoder may be received at an encoder-decoder attention layer of the decoder.

Each encoder and/or decoder of the transformer architecture may include a plurality (and/or cascade) of (for example, identical) blocks of (for example, a plurality of different) layers. The context vector may in each case be fed, for example, into the encoder-decoder attention layer of a decoder block.

The physiological signal may include a respiration curve, an electrocardiogram curve and/or a movement curve.

The physiological signal, for example the respiration curve and/or electrocardiogram curve, may be determined per instant of the MRI scan. For example, a respiratory phase and/or a cardiac phase (for example, systole or diastole) may be associated with each MRI scan instant.

Outputting the determined physiological signal may include outputting a movement curve of the patient.

The movement curve of the patient may include a movement of the patient during the MRI scan. For example, the patient may have convulsions while they are lying on a patient table and/or in a reception tube.

The movement curve may be used to adjust the MRI raw data (also: modify and/or correct) in order to obtain an MRI dataset that includes an MRI scan of a calm patient (for example a patient that is not moving and/or that is lying still).

The method may further include a step of outputting the determined physiological signal.

The output physiological signal may facilitate (for example in the absence of dedicated sensors) and/or improve monitoring of the vital signs of the patient.

The method may include a step of modifying, by the neural network, the received raw data by taking into account the determined physiological signal. Alternatively, or in addition, the method may include a step of outputting the modified raw data.

The physiological signal may vary over a period of the MRI scan. For example, the MRI scan may include a plurality of breathing cycles and/or a plurality of cardiac cycles. Alternatively, or in addition, the MRI scan may extend over a plurality of repetition times (TR) and/or a plurality of TR periods.

Modifying the raw data may include generating movement-adjusted raw data. Movement-adjusted raw data is raw data that does not exhibit any movement artifacts, or fewer artifacts than the original raw data. Modifying the raw data may include the raw data being converted into an equivalent to a predetermined physiological state (for example, a predetermined instant of a breathing cycle, a movement cycle and/or a cardiac cycle, also: cardiac phase for short).

One aspect relates to the use of the determined physiological signal in order to generate corrected raw data, for example movement artifact-adjusted raw data.

The method may include a step of receiving sensor data with regard to the physiological signal of the patient and/or with regard to a movement of the patient. The sensor data may have been recorded by a sensor during the creation of the MRI scan. Modifying the received raw data may further take into account the received sensor data.

Modifying (and/or correcting) the MRI raw data as a function of the physiological signal may be improved in that the physiological signal is additionally measured by (for example, dedicated) sensors. The measuring signal may be used to validate the determined signal, or vice versa. Alternatively, or in addition, sensor data may be received that directly or indirectly influences the physiological signal to be determined (for example, substance concentration, for example hormone concentration in the blood of the patient, that have a regulating effect on the breathing rate). Alternatively, or in addition, movement signals of the patient may be detected. Alternatively, or in addition, modifying (and/or correcting) the MRI raw data as a function of the received sensor data and/or the received movement signals of the patient may improve the quality of the MRI raw data in that it is mapped (and/or converted) to MRI raw data of a motionless patient (for example, a patient lying still). For example, a comparison with known standard measurements is consequently made possible and/or facilitated.

The raw data may include temporally sorted k-space lines and/or Fourier-transformed k-space lines (for example in the readout direction).

The temporally sorted k-space lines may be encoded by a positional embedding.

The Fourier transform of the (for example k-space) raw data may be an alternative representation of the data, by way of which, for example, phase responses along the readout direction may be visualized more easily and/or that may be interpreted more easily, by example by a neural network.

Encoding the raw data may include a position encoding and/or an association between a position in the k-space and a position in a slice stack of the MRI scan.

The position in the k-space may include a position in a k-space line (for example, as a one-hot encoding). Alternatively, or in addition, the position in the k-space may include a temporal position.

One-hot may include a group of bits that may each only assume a single high value 1, or otherwise the low value 0.

Encoding may take place, for example, independently for the real part and the imaginary part of complex MRI raw data. For example, the position in the k-space may be encoded in the real part and the position in the slice stack may be encoded in the imaginary part.

The transformer architecture may include a raw data encoder that receives the raw data as input data and outputs a raw data context vector. The transformer architecture may Alternatively, or in addition include at least one decoder that receives the raw data context vector, for example at an encoder-decoder attention layer (and/or a multi-head attention layer).

The output data of the at least one decoder may collectively be referred to as a sequence. The sequence may include the determined physiological signal (for example, a respiration curve, an electrocardiogram curve and/or a movement curve). Alternatively, or in addition, the sequence may include modified (and/or corrected) MRI raw data.

In addition to a context vector received by at least one encoder at an encoder-decoder attention layer, the input data of the at least one decoder may in each case include a (for example shifted) output signal, for example with position encoding. The position encoding and/or the (for example shifted) output signal may be received (and/or fed-in) at an input layer of the decoder.

The transformer architecture may further include a sensor signal encoder that receives sensor data of a physiological signal during the MRI scan as input data and outputs a sensor signal context vector.

The at least one decoder may be configured to receive the sensor signal context vector, for example at an encoder-decoder attention layer (and/or a multi-head attention layer).

For example, the transformer architecture may include two decoders, a sensor signal decoder and an MRI raw data decoder (for example configured for receiving a sensor signal context vector or a raw data context vector). In an alternative embodiment the at least one decoder may be configured to receive both the raw data context vector as well as the sensor signal context vector.

In the inference phase a weighted sum of the two (raw data and sensor signal) context vectors may be formed for the same position encoding and be supplied to the at least one decoder.

Alternatively, or in addition, MRI raw data (for example k-space data) and data of the physiological signal may be arranged in a matrix.

The at least one decoder may include a signal decoder that is configured to output the determined physiological signal. Alternatively, or in addition, the at least one decoder may include a raw data decoder that is configured to output the modified raw data by taking into account the determined physiological signal.

Each of the encoders and decoders of the transformer architecture may include an (for example self-) attention mechanism. For example, at least one multi-head attention block (and/or a multi-head attention layer) may be implemented in each encoder and/or decoder. Alternatively, or in addition, at least one masked multi-head attention block (and/or a masked multi-head attention layer) may be implemented in each decoder. A context vector may be received by an encoder in an encoder-decoder attention layer (and/or a multi-head attention layer) of one or each decoder.

At least one feed-forward block may be implemented in each encoder and/or decoder.

Each encoder and/or decoder may include a plurality of (for example repeating) blocks, for example six (N=6) blocks.

The neural network (for example the transformer architecture) may be trained to carry out the method according to the method aspect. Training may include a step of receiving a training dataset. The training dataset may include raw data of an MRI scan of a patient as well as a physiological signal of the patient measured during the MRI scan as the ground truth. The measured physiological signal may have been measured by at least one sensor. Alternatively, or in addition, the training dataset may be synthetically generated. The synthetically generated training dataset may include synthetically generated raw data as well as a synthetically generated physiological signal as the ground truth.

Training may further include a step of determining a physiological signal from the raw data of the MRI scan. A loss function may be optimized in the step of determining the physiological signal from the raw data of the MRI scan. The optimization of the loss function may include a comparison of the physiological signal determined from the raw data with the physiological signal measured as the ground truth.

Alternatively, or in addition, training may include a step of determining modified MRI raw data from the raw data of the MRI scan on the basis of the determined, and/or the measured, physiological signal of the patient. A loss function may be optimized in the step of determining the modified MRI raw data.

The loss function may include, for example, a Mean Squared Error (MSE); and/or —possibly up to overall standardization—equivalent to the L2 loss), a Mean Absolute Error (MAE); and/or—possibly up to overall standardization—equivalent to the L1 loss), Structural Similarity Index Measure (SSIM) and/or a Peak Signal-to-Noise Ratio (PSNR). The MSE, MAE and/or the SSIM may be determined per voxel (and/or per pixel). Alternatively, or in addition, the loss function may include an average of the MSE, MAE and/or of the SSIM per voxel (and/or per pixel). The optimization may be a minimizing of the loss function, for example of the MSE. Alternatively, or in addition, apart from the MSE, MAE, and for example SSIM, the PSNR may be suitable or be used as the loss function for comparison of image-space data or k-space data.

A sensor signal encoder may receive the measured physiological signal at an input layer. A raw data encoder may receive the MRI raw data at an input layer. Training may include that a loss function of a first (or raw data) context vector is optimized as the output of the raw data encoder and a second (or sensor signal) context vector is optimized as the output of the sensor signal encoder.

The two encoders may be trained simultaneously with matching MRI raw data and time series of physiological signals (for example from the same training dataset and/or associated with the same acquisition timeframe).

The loss function may alternatively, or in addition include a contrastive loss function. Alternatively, or in addition, the loss function may be selected such that similar “embeddings” are generated per (for example positive) pair (for example including MRI raw data and measured physiological signal for the same acquisition timeframe). The scalar product of the context vectors of the two encoders may be maximized for (for example positive) pairs (for example contained in the same training dataset) and otherwise minimized (for example if the raw data and the measured physiological signal pertain to different training datasets).

The contrastive loss function may be used, for example, with pairwise comparison, such as the context vectors as outputs of the two encoders. The contrastive loss function may also be referred to as a Siamese Network Loss. Alternatively, or in addition, the two encoders may also be referred to as Siamese Networks. The comparison may differ between positive pairs (for example, corresponding first and second or raw data and sensor signal context vector) and negative pairs (for example, non-corresponding first and second or raw data and sensor signal context vector). “Corresponding” may denote associated with the same MRI scan or the same acquisition timeframe here.

An optimization of the contrastive loss function may include that a spacing of a positive pair is minimized and a spacing of a negative pair is maximized (for example, in each case below or above a predetermined threshold value).

Training of the sensor signal encoder and of the raw data encoder may be frozen, for example after reaching (for example undershooting) a predetermined threshold value (also: optimization threshold) of the loss function. At least one decoder of the transformer architecture may subsequently be trained, for example using a k-space line in the MRI raw data.

The same k-space line in different physiological states (for example, as specially prepared datasets that may be included, for example, in the training data) may be used for training the at least one decoder. For example, test subject measurements and/or repetitions of scans may be carried out. Alternatively, or in addition, acquired training data may be re-sorted (for example, temporally, for example per breathing cycle, movement cycle and/or cardiac cycle, and/or spatially, for example as the permutation of slices). The acquired training data may, for example, be re-sorted such that a target dataset forms a dataset with physiological signal (physio- for short) data in a similar respiratory, movement and/or cardiac cycle state and the input datasets correspond to the datasets with regular physio-activity.

According to a further method aspect, a computer-implemented method for determining modified raw data of an MRI scan on the basis of a physiological signal of a patient is provided. The method includes the step of receiving raw data of an MRI scan of a patient. The method may further include the step of determining, by a neural network (NN), modified raw data using a physiological signal (for example, assumed and/or derived from the received raw data) of the patient from the received raw data. The NN includes a transformer architecture.

The method according to the further method aspect may include all features that are disclosed in the connection with the (for example first and/or preceding) method aspect.

According to an apparatus aspect, a neural network (NN) for determining a physiological signal of a patient using an MRI scan is provided. The NN may be implemented in an apparatus. The NN includes a receiving interface that is configured for receiving raw data of an MRI scan of a patient. The NN further includes a transformer architecture that is configured for determining a physiological signal of the patient from the received raw data.

According to a further apparatus aspect, an NN for determining modified raw data of an MRI scan on the basis of a physiological signal of a patient during an acquisition timeframe is provided. The NN includes a receiving interface that is configured for receiving raw data of an MRI scan of a patient. The NN further includes a transformer architecture that is configured for determining modified raw data using a physiological signal (for example, assumed and/or derived from the received raw data) of the patient during the acquisition timeframe from the received raw data.

The NN according to the (and/or the further) apparatus aspect may be configured to carry out the method according to the (and/or the further) method aspect. Alternatively, or in addition, the NN may include one or each feature that is disclosed in connection with the (and/or the further) method aspect.

According to a system aspect, a system for determining a physiological signal of a patient using an MRI scan is provided. The system includes an NN according to the (and/or according to the further) apparatus aspect and at least one MRI scanner.

According to a further aspect, a computer program product with program elements is provided that cause an NN to carry out the steps of the method for determining a physiological signal of a patient using an MRI scan, and/or for determining modified raw data of an MRI scan on the basis of a physiological signal of a patient, according to the (and/or the further) method aspect when the program elements are loaded into a memory of the NN.

According to yet a further aspect, a computer-readable medium is provided on which program elements are stored that may be read and executed by an NN in order to carry out steps of the method for determining a physiological signal of a patient using an MRI scan, and/or for determining modified raw data of an MRI scan on the basis of a physiological signal of a patient, according to the (and/or the further) method aspect when the program elements are executed by the NN.

The above-described properties, features and advantages and the manner in which they are achieved will become clearer and more understandable in light of the following description and the embodiments, that are explained in connection with the drawings. This following description does not limit the covered embodiments. Identical components or parts may be provided with identical reference numerals in different Figures. In general, the illustrations are not to scale.

### BRIEF DESCRIPTION OF THE DRAWINGS

FIG. 1 depicts a flowchart of a method for determining a physiological signal of a patient using an MRI scan according to an embodiment.

FIG. 2 depicts is a flowchart of training of an NN including a transformer architecture for determining a physiological signal of a patient (and/or for determining modified MRI raw data on the basis of a physiological signal of the patient during an acquisition timeframe) using an MRI scan according to the method of FIG. 1 according to an embodiment.

FIG. 3 depicts an overview of the structure and the construction of an NN including a transformer architecture that is configured for processing MRI raw data according to the method of FIG. 1 according to an embodiment.

FIG. 4 schematically depicts details of an encoder and decoder of the transformer architecture from FIG. 3, with input data and output data being shown in each case with, for example, a transmission of context vector from the encoder to the decoder according to an embodiment.

FIG. 5 diagrammatically depicts MRI raw data as input data and a physiological signal determined therefrom as output data of the NN including a transformer architecture from FIG. 3 according to an embodiment.

FIG. 6 diagrammatically depicts MRI raw data and an adjusted physiological signal as input data and modified MRI raw data as output data of the NN including a transformer architecture from FIG. 3 according to an embodiment.

### DETAILED DESCRIPTION

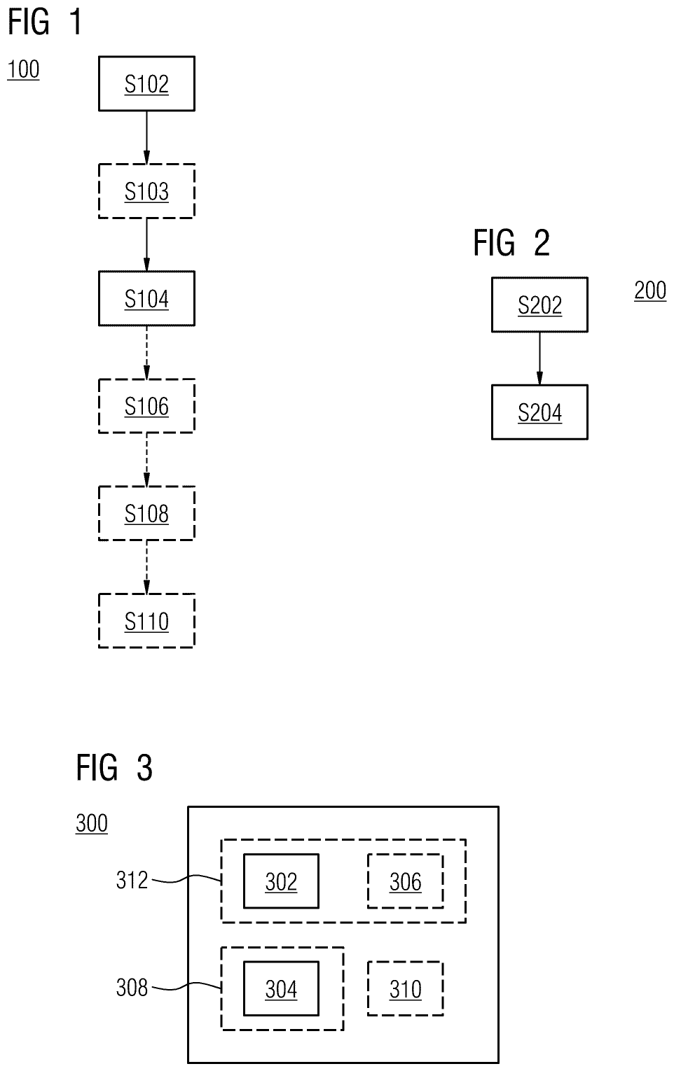

FIG. 1 schematically depicts a flowchart of a (for example computer-implemented) method 100 for determining a physiological signal of a patient using a magnetic resonance imaging (MRI) scan.

The method 100 includes a step S102 of receiving raw data of an MRI scan of a patient.

The method 100 may further include a step S104 of determining, by a neural network (NN), a physiological signal of the patient from the received S102 raw data. The NN includes a transformer architecture.

The method 100 may further include a step S106 of outputting the determined S104 physiological signal.

The method 100 may include a step S108 of modifying, by the NN, the received S102 raw data by taking into account the determined S104 physiological signal. Alternatively, or in addition, the method 100 may include a step S110 of outputting modified S108 raw data.

The method 100 may further include a step S103 of receiving sensor data with regard to the physiological signal of the patient (and/or with regard to a movement of the patient). The sensor data may have been recorded by a sensor during creation and/or during an acquisition timeframe) of the MRI scan.

Modifying S108 the received S102 raw data may take into account the received S103 sensor data.

FIG. 2 depicts training 200 of the NN, for example the transformer architecture, in order to determine in an inference phase a physiological signal of a patient and/or modified MRI raw data using an MRI scan.

Training 200 includes a step of S202 of receiving a training dataset. The training dataset includes raw data of an MRI scan of a patient and a physiological signal of the patient measured during the MRI scan as the ground truth. The measured physiological signal was measured by a sensor.

Training 200 may further include a step S204 of determining a physiological signal from the raw data of the MRI scan.

A loss function may be optimized in the step of determining S204 the physiological signal from the raw data of the MRI scan. Optimizing the loss function may include a comparison of the physiological signal determined S204 from the raw data with the physiological signal measured as the ground truth.

Alternatively, or in addition, training 200 may include a step of determining modified MRI raw data on the basis of the determined, and/or the measured, physiological signal of the patient.

FIG. 3 schematically depicts an embodiment of an NN 300 for determining a physiological signal of a patient using an MRI scan (and/or for determining modified MRI raw data on the basis of a physiological signal of the patient during an acquisition timeframe).

The NN 300 includes a receiving interface 302 that is configured for receiving raw data of an MRI scan of a patient. The NN 300 further includes a transformer architecture 304 that is configured for determining a physiological signal of the patient from the received raw data.

The NN 300 may further include an output interface 306. The receiving interface 302 and the output interface 306 may be realized by an input-output interface 312.

The receiving interface 302 and/or the input-output interface 312 may further be configured to receive sensor data with regard to a physiological signal of the patient.

The output interface 306 and/or the input-output interface 312 may be configured to output the determined physiological signal of the patient. Alternatively, or in addition, the output interface 306 and/or the input-output interface 312 may be configured to output the determined modified MRI raw data on the basis of a physiological signal of the patient during an acquisition timeframe.

The transformer architecture 304 may be realized on a processor 308. The NN 300 may further include a memory 310.

The NN 300 may be configured to carry out the method 100. Alternatively, or in addition, the NN 300 may be trained by the training 200.

A system (not shown) may include at least one MRI scanner and the NN 300.

The technique (for example including the method 100, the training 200 and/or the NN 300) may also be referred to as a technique for predicting physiological signals by transformer architecture.

Based on the original transformer architecture (as described by Ashish Vaswani et al. in “Attention Is All You Need”, arXiv: 1706.03762v7 [cs.CL], the content of that is incorporated herein by reference) and based on the developments achieved in recent years in the field of the Large Language Model (LLM), physiological curves from MRI raw data may be obtained by the technique with the aid of a (for example sequence-to-sequence) transformer architecture, for example without the aid of external sensors. The underlying idea is that physiological signals always influence the MRI raw data and, conversely, inferences about the underlying physiological effects are thus possible from patterns in the temporal course of the MRI raw data scan.

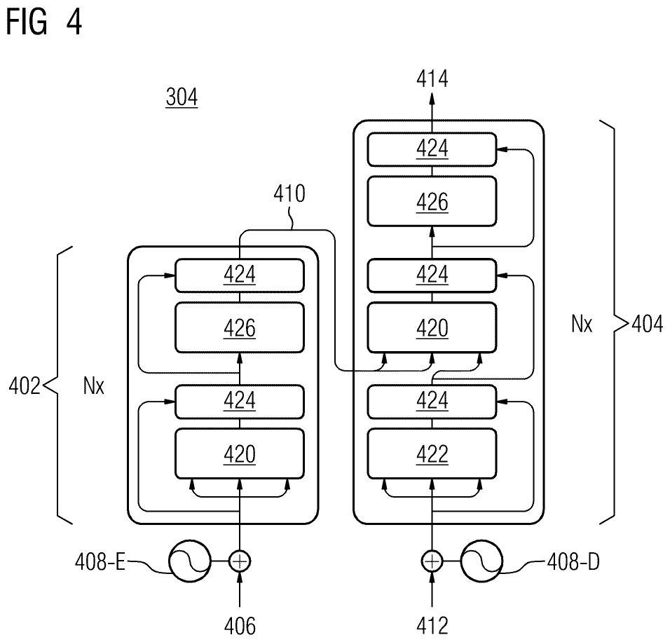

FIG. 4 represents a simple or fundamental diagram of a transformer architecture. In the encoder portion 402, a context vector 410 is constructed from input data 406 with positional encoding 408-E (with conventionally a series of words or data as the input data, however, MRI raw data 406-R; 406-I and/or sensor data 406-S, for example a physiological signal during the MRI acquisition timeframe, as the input data 406). The context vector 410 is passed to the decoder 404 that generates output data 414 (also: an output series) on the basis of the context vector 410. During the creation of the context vector 410, not only the immediately adjacent elements (for example, adjacent slices of the MRI scan and/or adjacent temporal instances of a time series of the MRI scan), but elements of the entire input data 406 (also: input series) identified as relevant in the training are incorporated in the determination (for example, calculation) of the continuation series. This is referred to as the attention mechanism and is the fundamental reason for why language models based on transformer architectures are far superior to those based on previous architecture that incorporate only the immediate vicinity in the prediction (for example, LSTM). The technique utilizes the attention mechanism for processing MRI raw data (for example with real part 406-R or imaginary part 406-I) and/or for processing a sensor signal 406-S, preferably a physiological signal during the MRI scan period.

The encoder blocks 402 and decoder blocks 404 shown in FIG. 4 are separable and may be recombined. Separating and recombining is conventionally used, for example, in architecture for language translation in order to make translation into different languages possible. In the context of the technique, separating and recombining the encoder blocks 402 and decoder blocks 404 makes processing and appropriate prior training of the respectively relevant blocks of MRI raw data 406-R; 406-I and/or sensor data 406-S possible for outputting a physiological signal 414-P determined from the MRI raw data and/or for outputting modified MRI raw data (for example with real part 414-R and imaginary part 414-I).

The positional encodings of the input data 406 or 412 of the encoder portion 402 and of the decoder portion 404 are schematically represented at reference numerals 408-E and 408-D. The input data 412 of the decoder portion 404 corresponds, for example to the right, for example with regard to a scanned slice (also: slice, and/or instance of a time series), to output data 414 generated (or shifted) in an immediately preceding implementation.

The encoder portion 402 schematically shown in FIG. 4 includes a cascade of NE (for example, six, NE=6) blocks each with multi-head attention layer 420, feed-forward layer 426 and Add&Norm layers 424.

The encoder portion 404 schematically shown in FIG. 4 includes a cascade of ND (for example, six, ND=6) blocks each with masked multi-head attention layer 422, multi-head attention layer 420, feed-forward layer 426 and Add&Norm layers 424.

The numbers NE and ND of encoder blocks or decoder blocks may be independent of one another. Alternatively, or in addition, a number of layers per encoder block or decoder block may be independent of a number of layers of a different (for example, encoder or decoder) block.

The context vector 410 output by the encoder portion 402 may be received at an encoder-decoder attention layer (for example, a multi-head attention layer) 420 of the decoder. The positional encoding (also: position encoding) 408-D, by contrast, is received in the input layer of the decoder portion 404.

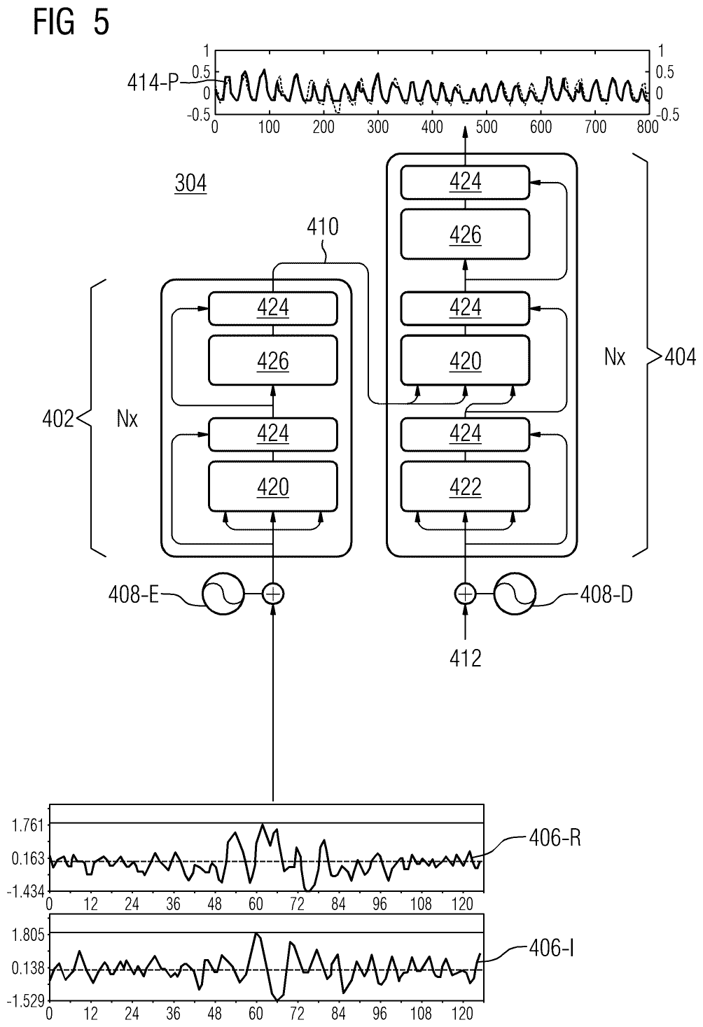

In a first embodiment, as shown by way of example in FIG. 5, the input data (also: input series) 406 (or the real and imaginary parts of the MRI raw data 406-R; 406-I) of the encoder 402, is composed of temporally sorted k-space lines or Fourier transforms of the k-space lines in the readout direction of the MRI scan. In an embodiment, the k-space-position of the k-space lines and their position in the layer stack are also encoded, for example in the form of a one-hot encoding 408-E. Alternatively, or in addition, the positional encoding mechanism at reference numeral 408-E may be modified such that, apart from the temporal position of the k-space line at the k-space position, the position in the layer stack may be taken into account. This may exploit the fact that the MRI data (for example the MRI raw data 406-R; 406-I) is in complex form and encoding may be carried out in both the real as well as the imaginary part. The output (also: output series) 414 of the decoder 414 may be composed of a physiological signal (physio-signal for short; also: physio-data) 414-P (for example, varying over an acquisition timeframe) (for example, of a respiration curve and/or a cardiac curve).

In an embodiment, the output (also: output series) 414 of the decoder 404 may include movement curves (for example as a further example of a physiological signal 414-P). The movement curves may be used as input data for existing algorithms that require the physio-data. This is represented by way of example at reference numeral 414-P in FIG. 5. Advantage of this embodiment is that external sensors are not required for patient measurements, and this makes simplified patient handling and resource savings (with regard to material, manufacturing expenditure and costs resulting therefrom) possible in production.

FIG. 5 depicts, by way of example, a physiological signal curve (broken lines) measured by an external sensor and a physiological signal curve 414-P (solid line) determined from the MRI raw data 406-R; 406-I.

The NN 300 including the transformer architecture 304 (also: model) may be trained by a large number of training datasets including MRI raw data files (for example, meas.dat-Files) 406-R; 406-S. If physiological sensors were active during the measurement, the sensor data 406-S may thus also be automatically in the MRI raw data files, so both input data 406; 406-R; 406-I (and for example 406-S) and output data 414; 414-P (and optionally 414-R; 414-I) may be extracted from a training dataset. This is particularly easy for respiratory curves since, in some newer (for example high-end) MRI scanners or MRI systems (for example, a 3 tesla MRI “Magnetom Vida”), a respiratory sensor is installed as standard in the spine coil that is used in most measurements.

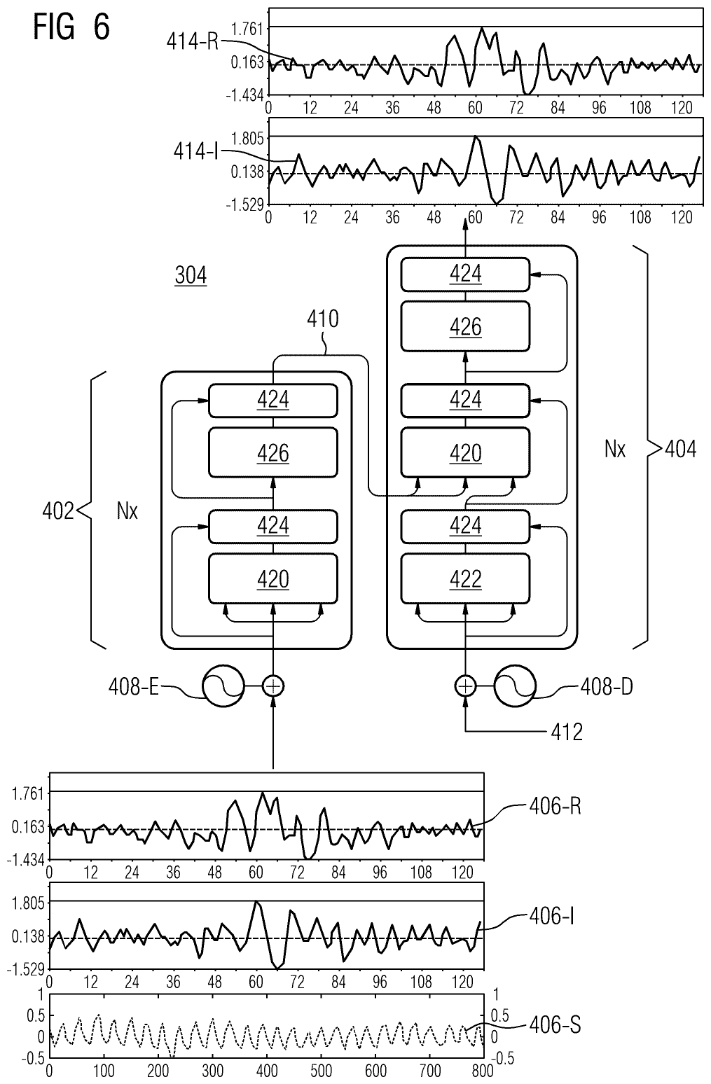

In a further embodiment, as schematically shown in FIG. 6, sorted k-space lines (or their Fourier transform) 406-R; 406-I may be used as input data (also: input series) 406; 406-R; 406-I in different physiological states. Data in an identical physiological state, for example modified (and/or corrected) MRI raw data 414-R; 414-I results as the output (also: output series) 414; 414-R; 414-I in the decoder in the embodiment. In a further embodiment, as outlined in FIG. 6 using the brackets, the input data (also: input series) 406; 406-R; 406-I; 406-S may include k-space lines (or their Fourier transform) 406-R; 406-I as well as physio-data (also: physiological signal) 406-S recorded or measured using sensors.

For training, first an encoder (also: sensor signal encoder) 402 for a physiological signal (also: physio-data) and an encoder (also: raw data encoder) 402 for MRI raw data may be provided. The two (for example raw data and sensor signal) encoders 402 may be trained simultaneously with matching MRI raw data 406-R; 406-I and physiological signals (also: physio-time series) 406-S from a training dataset. The loss function for generating the (for example raw data and sensor signal) context vectors 402 may be selected such that the two (for example raw data and sensor signal) encoders 402 for matching (also: positive) pairs (for example, MRI raw data 406-R; 406-I and physiological sensor signal 406-S relating to the same MRI scan and/or in the same training dataset) generate similar embeddings and/or context vectors 410, for example in that the scalar product of the two context vectors 410 for (for example positive) pairs is maximized and for other combinations (for example negative pairs and/or a context vector 410 relating to MRI raw data 406-R; 406-I and a context vector 410 relating to a physiological sensor signal 406-S for different MRI scans and/or in the different training datasets) is minimized. Training of the encoder block(s) 402 may subsequently be frozen.

Optimization of the contrastive loss function L_{C} (for example for the raw data and sensor signal context vectors 410) may include that a spacing dᵢ of a positive pair (xᵢ=0) is minimized and a spacing dᵢ of a negative pair (xᵢ=1) is maximized (for example, in each case below or above a predetermined threshold value), for example:

$${L}_{C} = \frac{1}{2 N} {\sum }_{i = 1}^{N} [ ( 1 - {x}_{i} ) \cdot {d}_{i}^{2} + {x}_{i} \cdot {( D - {d}_{i} )}^{2} ] .$$

The index i denotes here the pairs (for example of raw data and sensor signal context vectors 410) being considered whose total number is N, by way of example. D denotes here, by way of example, a threshold value of a spacing, above which pairs (for example of raw data and sensor signal context vectors 410) are regarded as being negative, and/or below which pairs (for example of raw data and sensor signal context vectors 410) are regarded as being positive.

The first term in the formula for the contrastive loss function L_{C} is non-vanishing only for positive pairs (for example of raw data and sensor signal context vectors 410). The second term in the formula for L_{C} is non-vanishing only for negative pairs (for example of raw data and sensor signal context vectors 410). In order to minimize the contrastive loss function L_{C}, the spacings dᵢ for positive pairs (for example of raw data and sensor signal context vectors 410) therefore have to be minimized, just like the difference D−dᵢ for negative pairs (for example of raw data and sensor signal context vectors 410), and this is equivalent to maximizing the spacing dᵢ for negative pairs (for example of raw data and sensor signal context vectors 410).

Specially prepared training datasets may be required for training the decoder-block 404, in which sets data with the same k-space line in different physiological states is available. The data in different physiological states may be generated, for example, by test subject measurements, in which the same scan is repeated many times. The data may be re-sorted such that a target dataset forms a dataset with physiological signal (also: physio-data) in a similar state and the input datasets correspond to the datasets with regular physiological signal (also: regular physio-activity). The decoder 404 may be trained to form the datasets with k-space lines in a similar physiological state using the context vectors of the regular datasets.

An inference phase may include MRI raw data (and/or k-space data) 406-R; 406-I as well as a physiological signal (also: physio-data) 406-S: either in that the data 406-R; 406-I; 406-S is combined in a matrix, or in that separate (for example, raw data and sensor signal) encoders 402 are implemented and both (for example raw data and sensor signal) context vectors 410 are transferred to the decoder 404, for example in a weighted sum.

A new context vector 410 is produced in the encoder 402 for each instant of the input data 406; 406-R; 406-I; 406-S. The decoder 404 therefore includes not just one context vector 410 per MRI scan, but all generated context vectors 410, as schematically outlined in FIGS. 4, 5 and 6 in each case by the arrow between the output of the encoder 402 and the encoder-decoder attention layer (for example, a multi-head attention layer) 402 of the decoder 404.

In conventional language processing, the resulting vector for each word after application of the attention mechanism is the context vector. This context vector in conventional language processing represents not only the original word, but also its context within the sentence, based on the relationships with other words. In language models, each context vector is typically between 128 bytes and 4,096 bytes long.

For the transformer architecture with MRI raw data 406-R; 406-I and optionally sensor signals 406-S (for example instead of words that are connected to form sentences), the length of the context vector 410 may similarly be between 128 bytes and 4,096 bytes long.

In patent application DE 10 2020 209 913 A1 and the patent specification U.S. Pat. No. 11,650,280 B2, phase correction raw data was obtained for the echoplanar imaging (EPI) from EPI raw data, whereby the scan could be spared from additional navigator data. In patent specification U.S. Pat. No. 11,650,280 B2, corrections were ascertained on the basis of fluctuations of a basic magnetic field in that over two or more short (for example TR) measuring periods (that are customarily only a few seconds long), in each case individual, at least one two-dimensional (2D) MRI datasets were generated. The individually generated MRI datasets of patent specification U.S. Pat. No. 11,650,280 B2 are very similar, and the differences in the data come about (for example, primarily) due to effects on the phase, for example due to breathing or field drift.

By contrast, according to the technique for determining a physiological signal of a patient using an MRI scan, (for example, primarily) measuring sequences, that over a fairly long time frame over a plurality of (for example, TR) periods “build up” one dataset per layer, are used. A classic example of a measuring sequence over a plurality of TR periods is the TSE sequence.

The following table compares a scan of six (6) k-space lines by the EPI imaging of patent specification U.S. Pat. No. 11,650,280 B2 and by the TSE sequence used, by way of example, in the technique for determining a physiological signal of a patient using an MRI scan. A complete k-space (schematically represented in the table in each case by -----) is produced with each TR period in the single-shot EPI scan, which k-space, independently of the other k-spaces, may be reconstructed to form an image. The complete k-space is only produced in the TSE sequence scan by combining the k-space lines gradually scanned over a plurality of TR periods (schematically represented in the table in each case by -----).

| EPI: | | | TSE: | | |
|---|---|---|---|---|---|
| TR 1 | TR 2 | TR 3 | TR 1 | TR 2 | TR 3 |
| - - - | - - - | - - - | - - - | | |
| - - - | - - - | - - - | | - - - | |
| - - - | - - - | - - - | | | - - - |
| - - - | - - - | - - - | - - - | | |
| - - - | - - - | - - - | | - - - | |
| - - - | - - - | - - - | | | - - - |

The technique may similarly be applied to EPI. However, in contrast to patent application DE 10 2020 209 913 A1 and patent specification U.S. Pat. No. 11,650,280 B2, the technique for determining a physiological signal of a patient using an MRI scan advantageously uses a transformer architecture.

Compared with the conventional architectures (used, for example, in DE 10 2020 209 913 A1 and U.S. Pat. No. 11,650,280 B2), for example including a Long Short-Term Memory (LSTM), the transformer architecture offers a plurality of advantages. For example, transformer architectures (also: transformer models) also identify correlations over long time segments. In the case of extraction of respiratory curves as physiological signals, this means, for example, that not only the last n (for example, where n is a single digit) breathing periods are continued, but by considering the entire (for example, respiratory curve) history it is also possible to identify patterns, such as: “as a rule, after a shortened breathing period, an extended breathing period occurred during the course of measurement” or “the amplitude of the respiration curve decreases over some periods and then suddenly increases again”. By contrast, the conventionally used LSTMs “forget” items of information from previous time segments, the further away from them they are.

Further, the self-attention mechanism in transformer architectures advantageously makes it possible to weight the importance of each part of the MRI raw data (for example, as a sequence) in different ways and adaptably. For example, short-term disruptions in the data quality due to movements or similar could be easily compensated in that greater use is made of previous data in the sequence. In conventional LSTMs by contrast, chaos ensures more quickly here since the most recently entered corrupt data is weighted more strongly.

Furthermore, relevant additional items of information about correlations of the data (for example, the layer position) may advantageously be easily encoded by way of modification of the positional encodings in transformer architectures. Conventionally used LSTMs know the temporal sequence but do not allow additional positional and contextual items of information to be easily integrated.

In an embodiment, the positional encoding may include items of slice information. For example, ten (10) slices with 128 k-space lines and 256 complex-valued samples each may be read out. Firstly the first line of the first slice, then the first line of the second slice, . . . (for example in each case the first line of the next slice), then the first line of the tenth slice, then the second line of the first slice, then the second line of the second slice, etc. (for example in each case the second line of the next slice, then in each case the third line up to the 128th slice) may be read out.

In the embodiment, the input data 406; 406-R; 406-I of the encoder is composed of all previously read-out k-space lines, including the information in which slice it was acquired. The conventional “positional encoding” describes the instant of the scan, with, for example, the very first acquired line in slice one being encoded with “1”, the first line in slice ten with “10”, the second acquired line in slice one with “11”, the second acquired line in slice ten with “20”, etc. Conventionally this occurs by way of modulation of a sine function or cosine function with the corresponding frequency.

The slice information may be co-modulated in different ways in order to obtain the positional encoding 408-E. The original positional encoding may be modulated; for example for the real part of the 256 samples, the slice position may be modulated in a similar manner for the imaginary part. Alternatively, or in addition, the slice position may be added as a constant offset to the original positional encoding. Furthermore, alternatively, or in addition, the slice position may be appended as an additional vector element, so, for example, an input sample includes 257=256 complex-valued samples with positional encoding+1 sample with slice position. Moreover, alternatively, or in addition the 256 complex-valued samples may be duplicated, so instead of two (2) channels with real and imaginary parts, four (4) channels are obtained. The original positional encoding may be applied to two of the four (for example, the first two) channels; the encoding of the slice position may be applied to the other two of the four (for example, the last two) channels.

Alternatively, or in addition to the slice position, further “items of position information” may be encoded in the positional encoding 408-E by the technique, for example the acquired contrast in scans in which a plurality of contrasts is simultaneously acquired (for example, with Dixon imaging or mapping sequences), the current echo train and/or data from (for example physiological) sensors and/or items of information about a detected movement (for example of the patient).

A physiological signal (also: items of physio information) 414-P may be extracted from MRI raw data 406-R; 406-I by the technique in order to transfer it into physio-curves (for example a respiration curve, a cardiac curve and/or a movement curve) and/or for correcting raw data.

The physiological signal (also: physio-data) may be determined without external sensors. This allows for improved patient handling, material saving, simplified and less expensive manufacture, and raw data correction (here optionally with the aid of external sensors).

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for determining a physiological signal of a patient using a magnetic resonance imaging (MRI) scan, the method comprising:
receiving raw data of the MRI scan of a patient; and
determining, by a neural network, a physiological signal of the patient from the received raw data, wherein the neural network comprises a transformer architecture.

2. The method of claim 1, wherein the physiological signal comprises at least one of a respiration curve, an electrocardiogram curve, or a movement curve.

3. The method of claim 1, further comprising:
outputting the determined physiological signal.

4. The method of claim 1, further comprising:
modifying, by the neural network, the received raw data taking into account the determined physiological signal; and
outputting the modified raw data.

5. The method of claim 4, further comprising:
receiving sensor data with regard to the physiological signal of the patient and/or with regard to a movement of the patient, wherein the sensor data was recorded by a sensor during creation of the MRI scan,
wherein modifying the received raw data further takes into account the received sensor data.

6. The method of claim 1, wherein the raw data comprises temporally sorted k-space lines or Fourier-transformed k-space lines.

7. The method of claim 1, wherein encoding of the raw data comprises position encoding and/or an association between a position in k-space and a position in a slice stack of the MRI scan.

8. The method of claim 1, wherein the transformer architecture comprises a raw data encoder which receives the raw data as input data and outputs a raw data context vector, wherein the transformer architecture comprises at least one decoder which receives the raw data context vector at an at least one encoder-decoder attention layer.

9. The method of claim 8, wherein the transformer architecture further comprises a sensor signal encoder that receives sensor data of a physiological signal during the MRI scan as input data and outputs a sensor signal context vector, wherein the at least one decoder receives the sensor signal context vector at one encoder-decoder attention layer at least.

10. The method of claim 8, wherein the at least one decoder comprises a signal decoder, outputs the determined physiological signal, and/or wherein the at least one decoder comprises a raw data decoder which outputs the modified raw data by taking into account the determined physiological signal.

11. The method of claim 1, wherein the neural network is trained by:
receiving a training dataset, wherein the training dataset comprises raw data of a magnetic resonance imaging, MRI, scan of a patient and a physiological signal of the patient measured during the MRI scan as ground truth, wherein the measured physiological signal is measured by a sensor;
determining a physiological signal from the raw data of the MRI scan,
wherein in the step of determining the physiological signal from the raw data of the MRI scan, an optimization of a loss function is executed, wherein the optimization of the loss function comprises a comparison of the physiological signal determined from the raw data with the physiological signal measured as the ground truth.

12. The method of claim 11, wherein a sensor signal encoder receives the measured physiological signal at an input layer, and wherein a raw data encoder receives the raw data at an input layer, and wherein the training comprises that a loss function of a sensor signal context vector is optimized as an output of the sensor signal encoder, and a raw data context vector as an output of the raw data encoder.

13. The method of claim 12, wherein the training of the sensor signal encoder and of the raw data encoder is frozen, for example after reaching an optimization threshold of the loss function, and wherein subsequently at least one decoder of the transformer architecture is trained using a k-space line in the raw data.

14. A neural network for determining a physiological signal of a patient using a magnetic resonance imaging, MRI, scan, comprising:
a receiving interface that is configured for receiving raw data of a magnetic resonance imaging, MRI, scan of a patient; and
a transformer architecture that is configured for determining a physiological signal of the patient from the received raw data.

15. The neural network of claim 14, wherein the physiological signal comprises a respiration curve, an electrocardiogram curve, and/or a movement curve.

16. The neural network of claim 14, wherein the neural network is further configured to output the determined physiological signal.

17. The neural network of claim 14, wherein the neural network is further configured to modify the received raw data by taking into account the determined physiological signal.

18. The neural network of claim 17, wherein the neural network is further configured to receive sensor data with regard to the physiological signal of the patient and/or with regard to a movement of the patient, wherein the sensor data is recorded by a sensor during creation of the MRI scan, wherein modifying the received raw data further takes into account the received sensor data.

19. The neural network of claim 14, wherein the transformer architecture comprises a raw data encoder which receives the raw data as input data and outputs a raw data context vector, wherein the transformer architecture comprises at least one decoder which receives the raw data context vector at an at least one encoder-decoder attention layer.

20. The neural network of claim 19, wherein the transformer architecture further comprises a sensor signal encoder that receives sensor data of a physiological signal during the MRI scan as input data and outputs a sensor signal context vector, wherein the at least one decoder receives the sensor signal context vector at one encoder-decoder attention layer at least.
